(19) United States
(12) Patent Application Publication
(10) Pub. No.: US 2013/0253613 A1
(43) Pub. Date: Sep. 26, 2013

(54) APPARATUS AND METHOD FOR ELECTRICAL STIMULATION USING HEADPHONE AUDIO

(71) Applicants: Davor Salahovic, Vaughan (CA); Sasa Marinkovic, Toronto (CA)

(72) Inventors: Davor Salahovic, Vaughan (CA); Sasa Marinkovic, Toronto (CA)

(21) Appl. No.: 13/430,372

(22) Filed: Mar. 26, 2012

(51) Int. Cl. A61N 1/36

(52) U.S. Cl. 607/61

(57) ABSTRACT

Transcutaneous Electrical Nerve Stimulation (TENS), a method of stimulating nerves using electrical current applied through the skin for therapeutic purposes, has been in use since the late 1970's, as have electronic units for self-administration of TENS therapy. With the recent increase in popularity of mobile devices capable of audio playback (smart phones, portable computing devices, MP3 players etc.), most TENS users already carry consumer electronic equipment capable of providing power and control to another device via its audio port. A TENS unit designed to: (a) be coupled with an audio playback capable device, (b) make use of its power and (c) rely on it for user interaction, provides a smaller, less expensive and more convenient portable treatment solution. This approach can be extended to other electrotherapy forms utilizing similar power budgets: Microcurrent Electrical Nerve Stimulation (MENS), Percutaneous Tibial Nerve Stimulation (PTNS), Electrical Muscle Stimulation/Neuromuscular Electrical Stimulation (EMS/NMES).

### CROSS-REFERENCE TO RELATED APPLICATIONS

N/A

### STATEMENT REGARDING FEDERALLY SPONSORED RESEARCH OR DEVELOPMENT

N/A

### THE NAMES OF THE PARTIES TO A JOINT RESEARCH AGREEMENT

Inventors: Davor Salahovic, Sasa Marinkovic.

### REFERENCES TO SEQUENCE LISTINGS, TABLES, COMPUTER PROGRAM LISTINGS

N/A

### FIELD OF THE INVENTION

The present invention relates generally to a battery-free electrotherapy solution and more particularly to a method for electrotherapy using another device's standard headphone audio output to provide power and control, and associated devices and software.

### BACKGROUND OF THE INVENTION

It has long been recognized that electrical nerve stimulation can have therapeutic effects, in particular in management of chronic pain. This recognition lead to scientific research and clinical trials, which, over the last four decades accumulated a body of evidence sufficient to indicate Transcutaneous Electrical Nerve Stimulation (TENS), as it is now formally known, in medical treatment and management of a number of conditions, related primarily, but not exclusively, to chronic pain. As medical research in the field of electrotherapy broadened, other specific electrotherapeutic methods were developed and standardized, today including but not being limited to: Microcurrent Electrical Nerve Stimulation (MENS), used in muscle and tendon repair and recovery, Percutaneous Tibial Nerve Stimulation (PTNS), indicated in the treatment of overactive bladder syndrome, and Electrical Muscle Stimulation, also known as Neuromuscular Electrical Stimulation (EMS/NMES), used in muscle toning, training, mobility and even cosmetic treatments. For brevity, TENS, MENS, PTNS and EMS/NMES will henceforth be referred to as Electrotherapy Devices (EDs). Modern research into applications of EDs continues to reveal unique benefits and advantages of different therapeutic methods, often citing minimal side effects, cases of efficacy where other approaches (e.g. pharmaceutical) either failed or were only partially successful, and the like.

Concurrent with these scientific advancements was the development of electronic equipment necessary to produce and administer electrical pulses for electrotherapy. There is now a broad variety of EDs on the market, ranging from sophisticated and highly programmable equipment intended for clinical sessions to considerably less expensive, user-owned devices that a patient can set up, self-administer and use regularly and without help. The latter, often labeled and marketed as “portable”, in fact have shortcomings which limit their true portability and popularity that would be expected, given the benefits.

Portable EDs on the market today rely on re-chargeable or disposable batteries. Because they are self-contained devices, often incorporating complex internal logic and user interfaces such as color Liquid Crystal Display (LCD) screens, their battery packs need to supply substantial power beyond that needed for the electrical pulses applied to the electrodes, and as such are invariably either large and heavy, or have short lives and hence require frequent replacement or recharging. Furthermore, the presence of an on-board user interface, most often a digital display, limits how small, truly portable and elegant today's EDs really are. This inherent bulkiness is the primary obstacle to ED use outside the home. The second obstacle to wider and more popular use of ED is the cost of batteries—financial in the case of disposable units or, equally problematic, the burden of inconvenience associated with re-charging in the case of reusable ones. Finally, the third limit to current ED usability is the “therapeutic device” image that a bulky, self-contained unit entails in public view, causing reluctance in the vast majority to use it outside one's home, although many public settings associated with waiting present very practical opportunities for electrotherapy. The current ED offering therefore leaves much to be desired in terms of power economy, true portability and user-friendliness. Hence, a lightweight, battery free solution that relied on power and functionality of existing portable electronic devices routinely carried, such as mobile phones, could bring benefits of electrotherapy to more users in more places with less cost, superior user experience and greater visual appeal and convenience than before.

### BRIEF SUMMARY OF THE INVENTION

User-portable devices specifically designed for self-administered electrical nerve stimulation today comprise dedicated power sources, user interfaces and control logic circuitry, all of which contribute to their size, weight and cost, while limiting their portability and visual appeal. This self-contained approach to their design has become redundant in an increasing user base already in possession of consumer electronic devices (mobile phones, personal computing devices, music players etc.) that are capable of lending power, control and user interaction to an electrical stimulation device. By relying on such consumer electronic devices to provide power and control functionality to an electrical nerve stimulation device, rather than having dedicated power and logic modules contained therein, that redundancy is overcome, with resulting benefits reflected in smaller size, reduced weight, reduced system complexity and reduced cost.

In accordance with an aspect of the present invention, there is provided an electronic device operable to provide, via electrodes, electrical nerve stimulation, using as the source of power another device's headphone audio output, of industry standard voltage and frequency range.

In accordance with another aspect of the present invention, there is provided an electronic device operable to provide, via electrodes, electrical nerve stimulation, using as the source of power another device's headphone audio output, and allowing the user a choice between multiple distinct stimulation timing patterns by means of a user-operable electrical switch.

In accordance with yet another aspect of the present invention, there is provided a method for controlling the timing, duration and amplitude of output signal in an electrical nerve stimulation device driven by another device' headphone audio output, by means of using purpose-built audio tracks in standard file formats on that audio playback device and its existing headphone audio volume control mechanism.

In accordance with yet another aspect of the present invention, there is provided a method for controlling the timing, duration and amplitude of output signal in an electrical nerve stimulation device driven by another device' headphone audio output, by means of executing software on the processor of that audio playback device. Other aspects and features of the present invention will become apparent to those of ordinary skill in the art upon review of the following description of specific embodiments of the invention with the accompanying figures.

### BRIEF DESCRIPTION OF THE SEVERAL VIEWS OF THE DRAWING

In the figures which illustrate by way of example only the embodiments of the present invention,

FIG. 1 shows a simplified diagram exemplary of an embodiment of the present invention in a typical user application scenario.

FIG. 2 shows a hardware block diagram of an electronic device, exemplary of an embodiment of the present invention.

FIG. 3 shows an idealized pulse timing diagram exemplifying a treatment pattern.

### DETAILED DESCRIPTION OF THE INVENTION

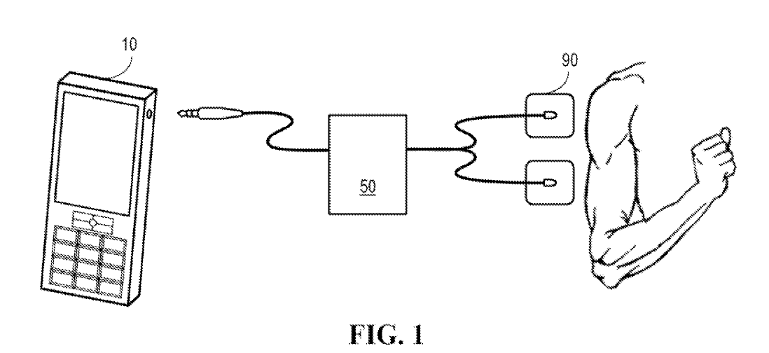

FIG. 1 exemplifies an embodiment of the invention in a typical user application, with an electronic device operable to provide electrical nerve stimulation 50 shown being connected to the headphone audio output of a mobile phone capable of music file playback (henceforth and without loss of generality “smart phone”) 10 and a pair of transcutaneous gel-contact electrodes 90 being used to apply electrical signal to the user's body via direct skin contact. In this embodiment of the invention, the smart phone 10 provides to the electrical stimulation device 50 all the electrical power necessary for internal operation of its circuits and the power necessary to generate output signals to the electrodes 90. A person of ordinary skill will readily appreciate that such electrical power need not come from a smart phone, but rather may be provided by any consumer electronic device capable of audio file playback to a pair of industry standard headphones, such as a personal music player, a tablet or laptop computer, or even an embedded audio subsystem, such as that in a massage chair or an airline seat. A person of ordinary skill will furthermore readily appreciate that the electrodes for stimulation need not be limited to those applied to the skin surface, rather, specialized electrodes applied to mucous membranes or penetrating the skin in a needle-like fashion are also possible, depending on the nature of the treatment. Furthermore, while the embodiment in FIG. 1 shows only one electrode pair, the invention may be embodied in a device operating multiple electrode pairs simultaneously, as dictated by the needs of the treatment and limited in principle only by the total available power budget.

Electrical nerve stimulation treatments require voltages, currents and slew rates above the operating capability of industry standard headphone audio outputs. For further clarity, values that exemplify signals in the embodiment depicted in FIG. 1, may be as follows:

2 Vₚ₋ₚ continuous sine wave of 1 kHz frequency for the audio signal,
+/−40 V square pulses of 50 microsecond duration, 200 millisecond period, for the electrodes.

The invention, therefore, must be embodied in a device capable of generating such higher values of instantaneous power, taking advantage of the premise that maximum average power available via headphone audio output exceeds average power demand of an electrical stimulation device.

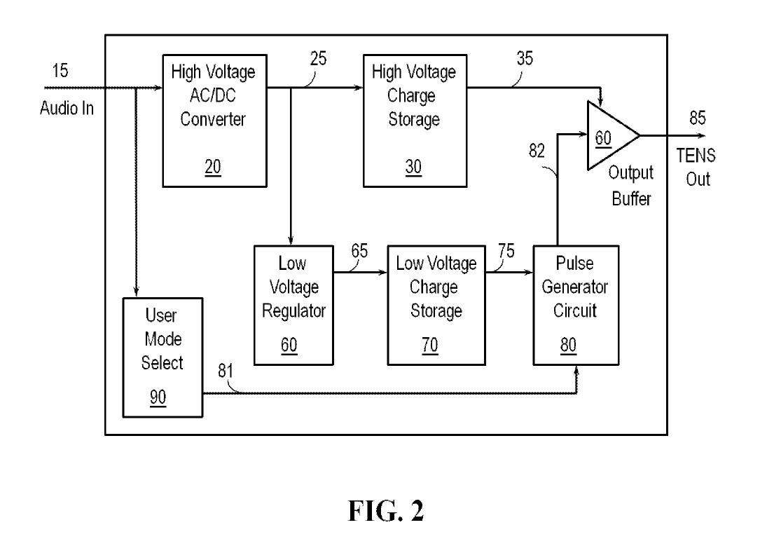

FIG. 2 shows a hardware block level diagram of an electronic device exemplary of an embodiment of the invention, with headphone audio signal 15 shown as an input and the electrode signal, in this particular case for TENS treatment, shown as the output, 85. In this embodiment, the low voltage AC signal arriving from the headphone audio output of an external device is used to generate a DC voltage of magnitude sufficient for use in electrical nerve stimulation. The high voltage AC/DC converter block, 20, serves to perform two functions to this end: the AC transformation to a higher peak voltage and energy-efficient signal rectification to achieve DC value essentially following the peak AC value. As will become apparent, thus rectified high-voltage power line, 25, is used by two other internal hardware blocks: 30 and 60.

In order to enable short electrical bursts of power far in excess of that provided by the continuous audio signal supply, a high voltage charge storage block, 30, is placed in the path of high voltage power, 25. A person of ordinary skill in the art will readily appreciate that such storage may be capacitive in nature and designed to be of sufficient capacity to cover the energy demand of one output burst, using the time between bursts for replenishment. The output of the high voltage storage block, 30, is a stable power rail, 35, capable of supplying required high voltage and unidirectional or bidirectional currents for the duration of the output power bursts.

The second use for the high-voltage power line 25 is the generation of a regulated, lower voltage power source for the device's internal circuitry. The reason for this separate, low voltage path is twofold: one, using a lower voltage to operate internal circuits saves power and two, internal circuitry powered from its own, regulated supply, remains immune to malfunction due to voltage fluctuations caused by the burst nature of the device's overall power load. In that sense, the low voltage regulator, 60, provides a DC rail, 65, at a fraction of the value of the high-voltage rail 25. The low voltage charge storage block, 70, provides voltage regulation to rail 65, yielding a stable low voltage DC rail 75.

Exemplified in this embodiment of the present invention, the use of a high voltage AC/DC converter block 20, as well as capacitive charge storage blocks 30 and 70, to create regulated high-voltage and low-voltage rails, 35 and 75, respectively, entirely covers the power requirements of the device, thus eliminating the need for batteries, so long as the device is connected to an active, industry standard headphone audio output of a smart phone, 15. Since, as will become apparent later, block 90 is entirely passive in nature, it requires no DC power supply for its operation.

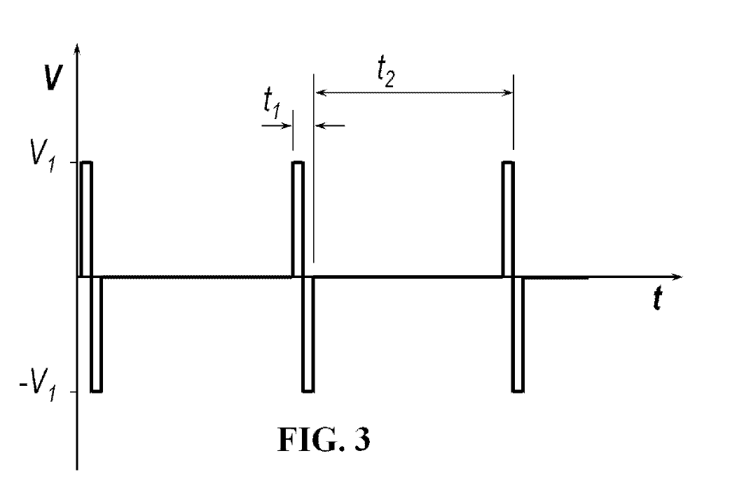

FIG. 3 exemplifies a timing diagram of a treatment pattern in transcutaneous electrical nerve stimulation. While absolute values of voltage V₁, pulse duration t₁ and pulse period t₂ vary in accord with the requirements of the treatment, it is generally true that the voltage V₁ is many times greater than the peak headphone audio output and that the pulse period t₂ is many times longer than the pulse duration t₁. In that sense, special circuitry within the device is needed and present to generate the pulse train matching the amplitude and timing requirements of the prescribed treatment program.

In FIG. 2, the pulse generator circuit, 80, serves to provide a low-voltage pulse train of desired timing, in one possible embodiment comprising a power efficient astable multivibrator circuit with a highly asymmetric duty cycle, dictated by nominal values of passive electronic components contained therein. Such an embodiment may include additional passive components of different values, instructed to be included in or excluded from the circuit by the user in real time (at time of device use), so as to generate multiple timing patterns, each consistent with a different treatment pattern. In the embodiment depicted in FIG. 2, this is achieved via user mode select block 90, operable to detect frequency or amplitude of the input audio signal and provide direction to pulse generator circuit 80 via control lines 81. In this arrangement, user mode selection is acquired by software executed on the smart phone, encoded by said software in the amplitude, frequency or both of the resulting audio signal 15, detected by the user mode select circuit 90 and translated into a timing pattern choice in the pulse generator circuit 80. A person of ordinary skill in the art will readily appreciate that such user selection can alternatively be achieved via a simple, user-operated, electrical multi-position switch, implemented in place of the user mode select block. The output signal, 82, of the pulse generator circuit 80 serves to meet the timing requirements of the signal driving the electrodes.

The voltage amplitude and current demand of the electrode output is met by the output buffer circuit 60. Exemplified in this embodiment, this block uses high voltage rail 35 as its primary source of power and creates output 85 by closely replicating, in that voltage, the timing sequence on the internally generated pulse train signal 82.

In an embodiment of the invention, the voltage rails 25, 35 and therefore the output 85 can be designed to follow proportionally the average peak value of the input audio signal 15. A person of ordinary skill will readily appreciate that the amplitude of the output signal 85 can be controlled by operating the volume control of the audio playback device. A person of ordinary skill will further readily appreciate that the peak audio signal value can be made stable and predictable by means of playing audio files of constant-amplitude, constant-frequency pure sine tones or that, conversely, playing audio files specifically composed to follow amplitude and frequency patterns can have those changes reflected in desired and predictable way in the electrical stimulation output signals. Furthermore, where the smart phone or other electronic device used for audio playback has a processor capable of executing software instructions, additional programming scenarios are possible, such as:

providing the user the interface and the options to create custom treatment programs by selecting from, combining or sequencing one or more audio tracks, resulting in predictable treatment patterns,
providing the user with interface and options to delay the start of the treatment to a desired time, set the treatment duration and course in advance,
store and re-use previously created and used treatment programs,
record history of use, and the like.

Of course, the above described embodiments are intended to be illustrative and are in no way limiting. The described embodiments of carrying out the invention are susceptible to many modifications of form, arrangement of parts, details and order of operation. The invention, rather, is intended to encompass all such modification within its scope, as defined by the claims.

What is claimed is:

1. An electronic device comprising:
a circuit operable to transform low voltage AC input signals of audible frequency range (20-20,000 Hz) into a higher DC voltage of magnitude suitable for therapeutic electrical nerve or muscle stimulation via electrodes applied to or through the skin, or to the mucous membrane (henceforth “high voltage DC supply”);
a capacitive storage for charge at said DC voltage sufficient to store the energy required for electrical pulses in said stimulation and additionally, the energy required to provide continuous power to other electronic circuits within the device, thereby eliminating the need for batteries;
a low power circuit operable to generate electrical pulses of specific duration and frequency, in accord with the timing pattern of the electrical stimulation treatment (henceforth “pulse generator circuit”);
an output circuit, operable to produce pulses of absolute voltage magnitude essentially matching that of the said high voltage DC supply and of timing essentially matching that of the said pulse generator circuit.

2. The device of claim 1, wherein the pulse generator circuit is operable to produce more than one timing pattern and includes an electrical switch by means of which a user may select the desired pattern.

3. The device of claim 1, wherein the AC input is provided by the industry standard headphone audio output of another portable electronic device comprising headphone audio playback capability (henceforth “user audio device”).

4. The device of claim 2, wherein the AC input is provided by the industry standard headphone audio output of a said user audio device.

5. The device of claim 3, further comprising in digital file format audio, the playback of which on the user audio device causes a desired waveform to appear at the headphone output to which the device is connected.

6. The device of claim 4, further comprising in digital file format audio, the playback of which on the user audio device causes a desired waveform to appear at the headphone output to which the device is connected.

7. The device of claim 3, further comprising application software executable on the user audio device by means of which the user can (a) select desired pulse patterns, (b) set treatment duration, (c) delay treatment start by or to a specific time, (d) select or program in advance the pulse amplitude, (e) create user-defined treatment courses consisting of a sequence of patterns of specific duration, (f) record treatment history.

8. The device of claim 4, further comprising application software executable on the user audio device by means of which the user can (a) select desired pulse patterns, (b) set treatment duration, (c) delay treatment start by or to a specific time, (d) select or program in advance the pulse amplitude, (e) create user-defined treatment courses consisting of a sequence of patterns of specific duration, (f) record treatment history.

9. A method of operating an electronic device having: a circuit operable to transform low voltage AC input signals of audible frequency range (20-20,000 Hz) into a higher DC voltage of magnitude suitable for electrical nerve or muscle stimulation via electrodes applied to the skin, a capacitive storage for charge at said DC voltage sufficient to store the energy required for electrical pulses in said stimulation and additionally, the energy required to provide continuous power to other electronic circuits within the device, a low power circuit operable to generate electrical pulses of specific duration and frequency, in accord with the timing pattern of the electrical nerve or muscle stimulation treatment, and an output circuit, operable to produce pulses of absolute voltage essentially matching that of the said high voltage DC supply, of desired polarity and of timing essentially matching that of the said pulse generator circuit,
said method comprising:
using the headphone audio output of a portable electronic device comprising headphone audio playback capability as the sole source of electric power for the operation of the device's internal circuitry and electrical pulses providing transcutaneous, transmucosal or percutaneous electrical nerve or muscle stimulation.

10. The method of claim 9, wherein the amplitude of the electrical pulses is adjustable by the user by means of controlling headphone output audio volume on the portable user audio device.

11. The method of claim 9, wherein the desired amplitude and frequency of headphone audio output on the portable user audio device to be used by the electrical stimulation device is achieved by means of playback of a digital audio file in standard industry formats, indistinguishable by the portable audio device from ordinary music playback and requiring no additional instruction code or user actions.

12. The method of claim 9, further executing software on programmable user audio devices to provide additional functions and controls to the user, namely to:
select desired pulse patterns,
set treatment duration,
delay treatment start by or to a specific time,
select or program in advance the pulse amplitude,
create user-defined treatment courses consisting of a sequence of patterns of specific duration, and
record treatment history.
